(19) United States
(12) Patent Application Publication
(10) Pub. No.: US 2024/0013923 A1
(43) Pub. Date: Jan. 11, 2024

(54) INFORMATION PROCESSING DEVICE, INFORMATION PROCESSING METHOD, AND PROGRAM

(71) Applicant: TERUMO KABUSHIKI KAISHA, Tokyo (JP)

(72) Inventors: Yoshihito MACHIDA, Sagamihara-shi (JP); Masaru IKEGAMI, Fujisawa-shi (JP); Nobuyuki TANIGAKI, Sagamihara (JP); Tomoko UEMURA, Hadano-shi (JP)

(73) Assignee: TERUMO KABUSHIKI KAISHA, Tokyo (JP)

(21) Appl. No.: 18/472,500

(22) Filed: Sep. 22, 2023

Related U.S. Application Data
(63) Continuation of application No. PCT/JP2022/010584, filed on Mar. 10, 2022.

(30) Foreign Application Priority Data
Mar. 23, 2021 (JP) 2021-049214

(51) Int. Cl. G16H 50/20; G16H 50/70

(52) U.S. Cl. CPC G16H 50/20; G16H 50/70; G16H 10/60

(57) ABSTRACT

An information processing device used in a system that predicts a prognosis of a patient by machine learning, the information processing device including: an input unit that receives an input of a plurality of sets of time-series data corresponding to a plurality of patients, the time-series data including a plurality of first parameters related to at least one of a condition and a treatment of each of the patients; and a processing unit that calculates an acquisition rate and an acquisition frequency of each of the first parameters included in the plurality of sets of time-series data, and selects a second parameter to be used for training data from the plurality of first parameters by using at least one of the calculated acquisition rate and the calculated acquisition frequency.

### CROSS-REFERENCES TO RELATED APPLICATIONS

This application is a continuation of International Application No. PCT/JP2022/010584 filed on Mar. 10, 2022, which claims priority to Japanese Application No. 2021-049214 filed on Mar. 23, 2021, the entire content of both of which is incorporated herein by reference.

### TECHNOLOGICAL FIELD

The present disclosure generally relates to an information processing device, an information processing method, and a program.

### BACKGROUND DISCUSSION

In recent years, it has been proposed to utilize a system using machine learning to predict the prognosis of a patient (see, for example, Japanese Patent Application Publication No. 2020-144471 A). A patient prognosis prediction model using machine learning is trained using data obtained by combining various parameters. For example, in the case of predicting the death of a patient entering an intensive care unit (ICU), basic information such as age and sex, disease information, vital value, medicine administration information, and the like are used as training data.

When training a prognosis prediction model using patient clinical data that was not acquired for machine learning, the frequency and combination of parameters actually acquired will vary depending on the patient's condition and disease. Therefore, even if a specific parameter is selected as a parameter for machine learning, there is a case where many parameters are not acquired. If the quality of data is relatively low because there are missing parameters used for training data, there is a concern that the relative accuracy of prediction by machine learning is reduced. Therefore, if it is attempted to use only the data of the patient having a relatively high degree of satisfaction of the parameter used for the training data, the number of sets of data that can be used as the training data is reduced, and sufficient training may not be performed. Further, if a parameter set suitable for machine learning cannot be acquired, the trained prediction model may not be applicable.

### SUMMARY

An information processing device, an information processing method, and a program are disclosed, which are capable of generating a required number of sets of training data with relatively high quality for performing machine learning from patient clinical time-series data.

An information processing device as one aspect of the present disclosure is an information processing device used in a system that predicts prognosis of a patient by machine learning, the information processing device including: an input unit that receives an input of a plurality of sets of time-series data corresponding to a plurality of patients, the plurality of sets of time-series data including a plurality of first parameters related to at least one of a condition and a treatment of each of the patients; and a processing unit that calculates an acquisition rate and an acquisition frequency of each of the first parameters included in the plurality of sets of time-series data, and selects a second parameter to be used for training data from the plurality of first parameters by using at least one of the calculated acquisition rate and the calculated acquisition frequency.

As an embodiment, the acquisition rate indicates a ratio at which the first parameter is included in the plurality of sets of time-series data.

As an embodiment, the acquisition frequency indicates a frequency at which the first parameter is included in data within a predetermined period of the time-series data.

As an embodiment, in a case where at least one of the acquisition rate and the acquisition frequency of the first parameter exceeds a predetermined threshold, the processing unit selects the first parameter as the second parameter to be used for the training data.

As an embodiment, a threshold of the acquisition frequency is different among the plurality of first parameters.

As an embodiment, the threshold of the acquisition frequency is determined on the basis of the number of sets of the time-series data including the first parameter exceeding the threshold.

As one embodiment, the plurality of sets of time-series data includes a first time-series data group and a second time-series data group, and the processing unit executes processing of selecting the second parameter from data obtained by the first time-series data group and the second time-series data group combined into one, and processing of individually selecting the second parameter from the first time-series data group and the second time-series data group.

As one embodiment, the input unit further receives an input of additional information including at least one of an initial symptom, an individual attribute, and a disease for each patient among the plurality of patients, and the processing unit executes processing of grouping the time-series data into a plurality of groups on the basis of the additional information and selecting the second parameter for each group among the plurality of groups.

As an embodiment, the processing unit increases the predetermined period for calculating the acquisition frequency as time elapses.

As an embodiment, the processing unit generates training data by using the selected second parameter.

As an embodiment, the processing unit generates the training data in a data format based on the acquisition frequency of the selected second parameter.

As an embodiment, the processing unit generates a learned model for predicting prognosis of a patient using the training data.

As an embodiment, for each of a plurality of provisional thresholds, in a case where the at least one of the acquisition rate and the acquisition frequency of the first parameter exceeds the provisional threshold, the processing unit selects the first parameter as a provisional parameter to be used for the training data, generates the training data and test data using the provisional parameter, generates a learned model for predicting prognosis of a patient using the training data, performs processing of determining accuracy of the learned model using the test data, and selects the provisional parameter with the determined highest accuracy as the second parameter.

As an embodiment, the time-series data includes at least any one of administration information of a medicine, a vital value, examination information, finding information, water intake information, water loss information, and treatment information.

As an embodiment, the administration information of the medicine includes at least any one of information of a type, an administration route, a dose, and an administration rate of an administration medicine.

As an embodiment, the vital value includes at least any one of information of a body temperature, a blood pressure, a heart rate, a respiratory rate, a pulse rate, oxygen saturation, a weight value, a central venous pressure, and an oxygen concentration during inhalation.

As an embodiment, the examination information includes at least any one of blood examination data, blood gas data, a urine examination, an electrocardiogram, and a diagnostic imaging result.

As an embodiment, the finding information includes at least any one of information of congestion, cyanosis, and a level of consciousness.

As an embodiment, the water intake information includes at least any one of information of a water intake amount and an infusion amount.

As an embodiment, the water loss information includes at least any one of information of a urine amount and a blood loss amount.

As an embodiment, the treatment information includes at least any one of information of introduction of a dialysis device, disengagement of the dialysis device, setting of the dialysis device, introduction of a ventilator, disengagement of the ventilator, and setting of the ventilator.

An information processing method as one aspect of the present disclosure is an information processing method executed by an information processing device used in a system that predicts prognosis of a patient by machine learning, the information processing method including: acquiring a plurality of sets of time-series data corresponding to a plurality of patients, the plurality of time-series data including a plurality of first parameters related to at least one of a condition and a treatment of each of the patients; calculating an acquisition rate and an acquisition frequency of each of the first parameters included in the plurality of sets of time-series data; and selecting a second parameter to be used for training data from the plurality of first parameters using at least one of the calculated acquisition rate and the calculated acquisition frequency.

A program as one aspect of the present disclosure is a non-transitory computer-readable medium storing a computer program that causes an information processing device to execute information processing executed by the information processing device used in a system that predicts prognosis of a patient by machine learning, the information processing including: acquiring a plurality of sets of time-series data corresponding to a plurality of patients, the plurality of sets of time-series data including a plurality of first parameters related to at least one of a condition and a treatment of each of the patients; calculating an acquisition rate and an acquisition frequency of each of the first parameters included in the plurality of sets of time-series data; and selecting a second parameter to be used for training data from the plurality of first parameters using at least one of the calculated acquisition rate and the calculated acquisition frequency.

According to the present disclosure, since the second parameter to be used for the training data is selected using the acquisition rate and the acquisition frequency of the first parameter included in the time-series data, it is possible to generate the necessary number of sets of training data with high quality for performing the machine learning from the patient clinical time-series data.

### BRIEF DESCRIPTION OF THE DRAWINGS

FIG. 1 is a block diagram illustrating a schematic configuration of an information processing device according to an embodiment.

FIG. 2 is a functional block diagram illustrating an example of a schematic configuration of a processing unit in FIG. 1.

FIG. 3 is a diagram illustrating an example of time-series data related to one patient.

FIG. 4 is a diagram illustrating an example of an acquisition rate and an acquisition frequency calculated from a plurality of sets of time-series data.

FIG. 5 is a diagram illustrating an example of training data generated from time-series data of one patient.

FIG. 6 is a flowchart illustrating an information processing method according to an embodiment.

FIG. 7 is a flowchart illustrating an information processing method according to another embodiment.

### DETAILED DESCRIPTION

Configuration of Information Processing Device

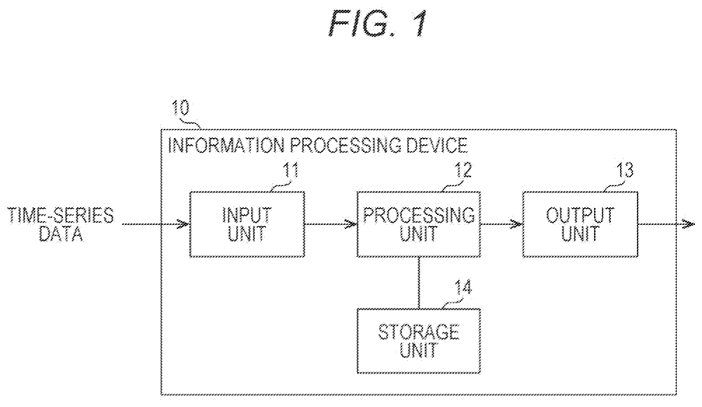

Set forth below with reference to the accompanying drawings is a detailed description of embodiments of an information processing device, an information processing method, and a program. An information processing device 10 according to an embodiment can be used for selection of a parameter to be used for training data in a system that predicts prognosis of a patient by machine learning. The information processing device 10 can use a computer such as a personal computer (PC) and a workstation. The information processing device 10 may be disposed, for example, in a medical institution such as a hospital, an information processing facility that aggregates information from a plurality of medical institutions, or the like. As illustrated in FIG. 1, the information processing device 10 includes an input unit 11, a processing unit 12, an output unit 13, and a storage unit 14.

The input unit 11 is a portion where the information processing device 10 receives an input of time-series data. In a case where the information processing device 10 receives the time-series data from another device via a communication line, the input unit 11 includes a communication interface with the other device. In a case where the information processing device 10 acquires time-series data stored in a storage medium such as a magnetic storage medium, a magneto-optical storage medium, or an optical storage medium, the input unit 11 may include a reading device of the storage medium.

The time-series data can include time-series information for a plurality of parameters related to at least one of a condition and a treatment of each of the patients acquired at the clinical time. A parameter included in the time-series data is set as a first parameter. One set of time-series data may include information from before treatment to the end of treatment for one patient. The input unit 11 receives an input of a plurality of sets of time-series data to be used for machine learning. Hereinafter, a plurality of sets of time-series data related to a plurality of patients may be referred to as a time-series data group. The time-series data group may include, for example, time-series data of the order of hundreds, thousands, or tens of thousands.

The time-series data may include at least any one of information of administration information of a medicine, a vital value, examination information, finding information, water intake information (water IN information), water loss information (water OUT information), and treatment information for one patient.

In one embodiment, the administration information of the medicine may include at least any one of information of a type, an administration route, a dose, and an administration rate of the administration medicine.

In one embodiment, the vital value may include at least any one of information of a body temperature, a blood pressure, a heart rate, a respiratory rate, a pulse rate, oxygen saturation, a weight value, a central venous pressure, and an oxygen concentration during inhalation.

In one embodiment, the examination information may include at least any one of information of blood examination data, blood gas data, a urine examination, an electrocardiogram, and a diagnostic imaging result.

In one embodiment, the finding information may include at least any one of information of congestion, cyanosis, and level of consciousness.

In one embodiment, the water intake information may include at least any one of information of a water intake amount and an infusion amount.

In one embodiment, the water loss information may include at least any one of information of a urine amount and a blood loss amount.

In one embodiment, the treatment information may include at least any one of information of introduction of the dialyzer, disengagement of the dialyzer, and setting of the dialyzer, and introduction of the ventilator, disengagement of the ventilator, and setting of the ventilator.

Each parameter of the time-series data can be adopted as an explanatory variable of machine learning. A part of the time-series data may be an objective variable of machine learning. For example, the introduction of a dialysis device and the introduction of a ventilator included in the treatment information can be prognosis (outcome) to be predicted, and thus can be an objective variable.

The processing unit 12 executes various arithmetic processing. The processing unit 12 includes one or more processors and memories. The “processor” can be, for example, a general-purpose processor, a dedicated processor specialized for a specific process, or the like, but is not limited to a general-purpose processor or a dedicated processor specialized for a specific process. The general-purpose processor can read the program stored in the memory and execute processing according to the program. The processing executed by the processing unit 12 will be described below.

The output unit 13 outputs a result of the processing by the processing unit 12 to the outside of the information processing device 10. The output unit 13 may include a communication interface for another system. The output unit 13 may include a writing device for storing information in a storage medium such as a magnetic storage medium, a magneto-optical storage medium, or an optical storage medium. The information processing device 10 may include a storage device in the information processing device 10.

The storage unit 14 can store information necessary for processing performed by the processing unit 12, information generated by the processing unit 12, a program executed by the processing unit 12, and the like. The storage unit 14 may be configured using, for example, any one or more of a semiconductor memory, a magnetic memory, an optical memory, and the like. The semiconductor memory may include a volatile memory and a nonvolatile memory. The magnetic memory may include, for example, a hard disk, a magnetic tape, and the like.

Configuration of Processing Unit

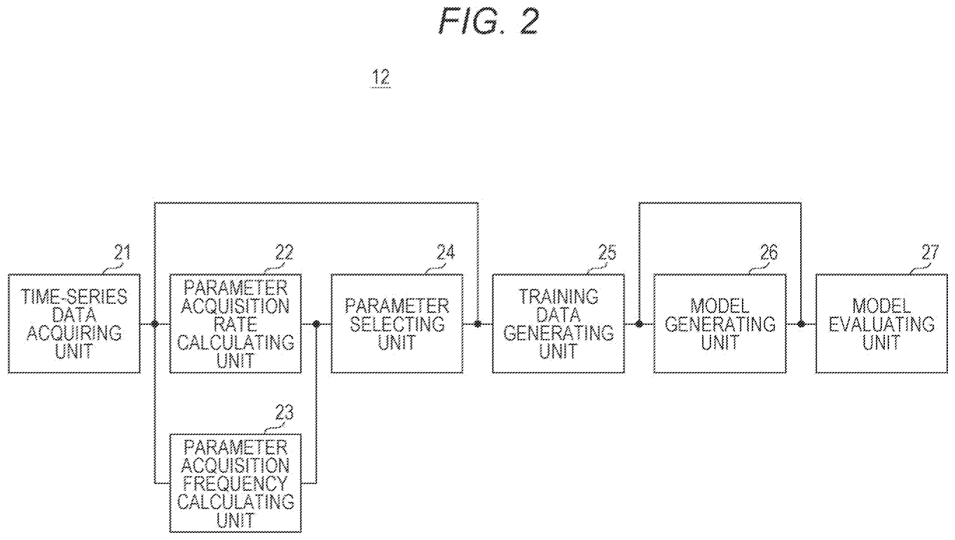

As illustrated in FIG. 2, the processing unit 12 includes a time-series data acquiring unit 21, a parameter acquisition rate calculating unit 22, a parameter acquisition frequency calculating unit 23, and a parameter selecting unit 24. The processing unit 12 may further include a training data generating unit 25, a model generating unit 26, and a model evaluating unit 27. Each unit of the processing unit 12 may be a hardware module or a software module. The function of each component of the processing unit 12 is executed by the processing unit 12.

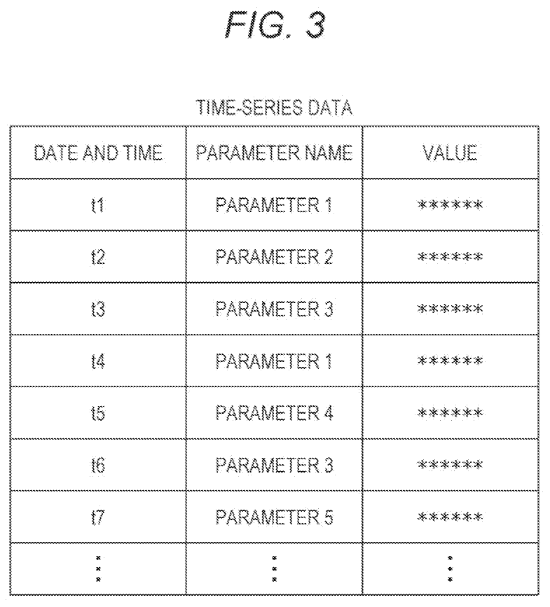

The time-series data acquiring unit 21 is configured to acquire time-series data via the input unit 11. The time-series data includes information such as clinical examination and treatment of a patient collected, for example, in a medical institution such as a hospital. A simplified example of time-series data for one patient is shown in FIG. 3. The “date and time” is information indicating the date and time when the data of each parameter included in the time-series data was acquired. The “parameter name” includes a name of each parameter or information for identifying each parameter. The “value” is information indicating the value of the parameter specified by the parameter name. As an example, “date and time”, “parameter name”, and “value” include information such as “Mar. 1, 2021, 10:10”, “blood pressure”, and “130”, respectively. The format of the time-series data as illustrated in FIG. 3 is merely an example. The time-series data can also be data collected in time series for each parameter.

The parameter acquisition rate calculating unit 22 is configured to calculate an acquisition rate of each parameter included in the plurality of sets of time-series data. The acquisition rate indicates a rate at which each parameter is included in the plurality of sets of time-series data. For example, in a case where the number of sets of time-series data is 1000 and the number of sets of time-series data including a specific parameter is 900 of the number of sets of time-series data, the acquisition rate of the parameter is 90%. The acquisition rate can be a rate at which the target parameter is included in the entire time-series data. The acquisition rate may be a rate at which a target parameter is included in the time-series data within a predetermined period.

The parameter acquisition frequency calculating unit 23 is configured to calculate the acquisition frequency of each parameter included in the plurality of sets of time-series data. The acquisition frequency indicates a frequency at which a target parameter is included in data within a predetermined period of time-series data including the target parameter. For example, when the predetermined period is one day and the number of times the target parameter is included in the time-series data during the period is four, the acquisition frequency is four. The parameter acquisition frequency calculating unit 23 may calculate the acquisition frequency of each parameter for each time-series data. The parameter acquisition frequency calculating unit 23 may calculate an average acquisition frequency, which is an average value of the acquisition frequencies of each parameter, for all sets of the time-series data. Further, the parameter acquisition frequency calculating unit 23 may calculate an acquisition frequency standard deviation that is a standard deviation of the acquisition frequency.

The parameter selecting unit 24 is configured to select a plurality of parameters to be used for training data by using at least one of the acquisition rate and the acquisition frequency calculated by the parameter acquisition rate calculating unit 22 and the parameter acquisition frequency calculating unit 23. The plurality of parameters used for the training data is second parameters. In a case where at least one of the acquisition rate and the average acquisition frequency of the parameter exceeds a predetermined threshold, the parameter selecting unit 24 selects the parameter as a parameter to be used for the training data. The parameter selected by the parameter selecting unit 24 corresponds to an explanatory variable in machine learning.

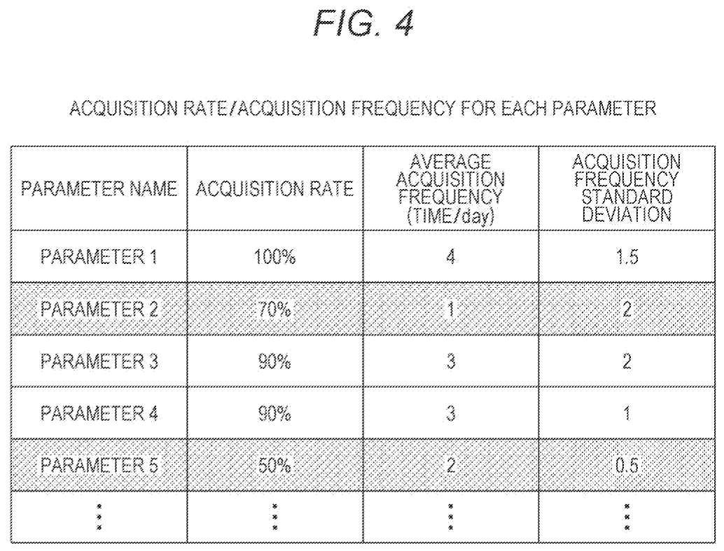

For example, it can be assumed that the acquisition rate and the average acquisition frequency for each parameter are illustrated in FIG. 4. For example, it can be assumed that the parameter selecting unit 24 sets the threshold of the acquisition rate to 80% and the threshold of the average acquisition frequency to 2, and selects a parameter exceeding both the thresholds. In this case, since the parameters 1, 3, and 4 in FIG. 4 exceed these thresholds, they are selected as the second parameters. Parameters 2 and 5 are not adopted as the second parameter.

The acquisition frequency standard deviation may be considered in the selection of the parameter. When the standard deviation is relatively large even with the same average acquisition frequency, the parameter acquisition frequency has a larger variation than when the standard deviation is relatively small. Therefore, even if the same threshold is set, the number of sets of data exceeding the threshold may be smaller as the standard deviation is larger. Therefore, a parameter with a smaller acquisition frequency standard deviation may be selected in preference to a parameter with a larger acquisition frequency standard deviation.

According to the characteristic of each parameter, the threshold of the average acquisition frequency may be different for each parameter of the plurality of parameters. For example, the blood examination data can be collected once a day, the blood pressure can be measured three times a day, and the like, which can be set according to the frequency that can be performed in actual clinical practice.

In addition, only time-series data in which the acquisition frequency of the selected parameter exceeds the threshold of the acquisition frequency can be used as the training data for machine learning. The threshold of the acquisition frequency may be determined in consideration of the number of sets of time-series data of the acquisition frequency exceeding the threshold. When the threshold is set to be relatively low, there is a possibility that data of the parameter is often missing and high-quality training data is not obtained. However, if the threshold is set too high, a sufficient number of sets of time-series data are not included, and thus, there is a possibility that the number of sets of training data necessary for performing machine learning cannot be secured. For example, the threshold may be set so that the number of sets of time-series data exceeding the threshold is equal to or larger than a predetermined number (for example, 10,000). Furthermore, for example, the threshold may be set so that a predetermined ratio (for example, 90%) or more of all sets of time-series data exceed the threshold.

The parameter selecting unit 24 may select some or all parameters as parameters to be used for the training data from among a plurality of parameters in which at least one of the acquisition rate and the average acquisition frequency of the parameters exceeds a predetermined threshold. The parameters to select may be determined depending on the prognosis (outcome) to be predicted. A plurality of parameters that can be selected depending on the prognosis to be predicted may be stored in the storage unit 14.

In one embodiment, the processing unit 12 may output the plurality of parameters selected by the parameter selecting unit 24 to another device via the output unit 13. The processing of the training data generating unit 25, the model generating unit 26, and the model evaluating unit 27 described below may be executed by another device.

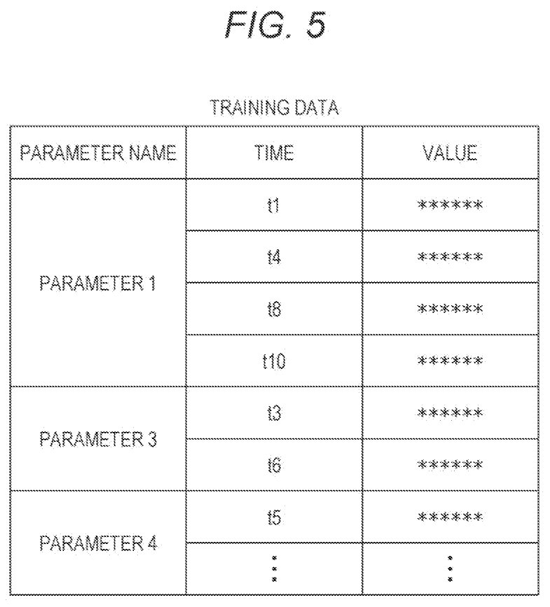

The training data generating unit 25 generates a plurality of sets of training data for machine learning by using the parameters selected by the parameter selecting unit 24 in the time-series data. For example, as illustrated in FIG. 4, in a case where the parameter 2 and the parameter 5 among the parameter 1 to the parameter 5 are not adopted, the parameters 2 and 5 are not included in the training data. In this case, the training data includes data indicating the values of the time series of the parameter 1, the parameter 3, and the parameter 4 as illustrated in FIG. 5 in a simplified manner. One training data may include data from before the start of treatment to the end of treatment of one patient for the selected parameter. The training data of FIG. 5 is an example. The training data can have various formats.

The training data generating unit 25 may generate the training data in a data format based on the acquisition frequency of each selected parameter. For example, for a parameter acquired every hour, the training data may be in a format in which a total of 24 values are stored every hour per day. On the other hand, in a case where the average acquisition frequency is a parameter of 3 times per day, the training data generating unit 25 can set the training data to a format in which three sets of data are stored per day. By doing so, it is possible to reduce data loss in the training data, so that the machine learning can be performed without requiring correction processing or with a small number of algorithms.

The training data generating unit 25 may generate test data for verifying the accuracy of the learned model generated by machine learning in addition to the training data. For example, the training data generating unit 25 can set a predetermined ratio of data generated from the time-series data of the parameter selected by the parameter selecting unit 24 as the training data and the rest as the test data. The predetermined ratio may be, for example, 80% or the like.

The training data generating unit 25 may pass the generated training data and test data to the model generating unit 26 and the model evaluating unit 27, respectively, in order to generate a learned model.

In one embodiment, the processing unit 12 may output the training data and the test data generated by the training data generating unit 25 to another device via the output unit 13 in order to generate the learned model by another device. The processing of the model generating unit 26 and the model evaluating unit 27 described below may be executed by another device.

The model generating unit 26 generates a learned model for predicting the prognosis of the patient using the training data generated by the training data generating unit 25. The prognosis of the patient can be rephrased as an outcome. The outcome includes information such as the life or death of the patient after treatment, whether or not the artificial dialysis has been introduced, whether or not a ventilator has been introduced, the number of days of stay in the ICU when the patient enters the ICU, the severity score of the patient after treatment, the presence or absence of complications, and the blood pressure and heart rate of the patient after treatment. The outcome corresponds to an objective variable in machine learning.

The model evaluating unit 27 is configured to evaluate the prediction accuracy of the learned model generated by the model generating unit 26 using the test data generated by the training data generating unit 25. Therefore, first, the model evaluating unit 27 predicts an outcome using the learned model and the test data. Next, the model evaluating unit 27 calculates the prediction accuracy from the degree of coincidence between the predicted outcome and the actual outcome.

In one embodiment, the prediction accuracy by the model evaluating unit 27 may be fed back to the setting of the threshold in the parameter selecting unit 24. The processing unit 12 may determine a threshold at which the best prediction accuracy can be obtained by machine learning as the threshold in the parameter selecting unit 24.

For example, a plurality of provisional thresholds can be prepared in advance in the processing unit 12. For each of the plurality of provisional thresholds, in a case where at least one of the acquisition rate and the acquisition frequency of each parameter exceeds the provisional threshold, the parameter selecting unit 24 selects the parameter as a provisional parameter to be used for the training data. The training data generating unit 25 generates training data and test data by using the selected provisional parameters. The model generating unit 26 generates a learned model for predicting the prognosis of the patient using the training data. The model evaluating unit 27 performs processing of determining the accuracy of the learned model using the test data.

After performing the above processing on all the provisional thresholds, the processing unit 12 selects a provisional parameter having the highest determined accuracy as a parameter for performing machine learning. In addition, the processing unit 12 adopts a learned model corresponding to the selected parameter as a learned model for predicting the prognosis of the patient.

Processing of a plurality of Time-Series Data Groups

The information processing device 10 may acquire a time-series data group including a plurality of sets of time-series data, for example, from a plurality of medical institutions such as hospitals. The processing unit 12 may execute both processing of selecting an acquisition rate and a parameter from one time-series data group that combines a plurality of time-series data groups and processing of selecting a parameter from each time-series data group as an individual time-series data group. For example, it can be assumed that the processing unit 12 acquires a first time-series data group from a first medical institution and a second time-series data group from a second medical institution as time-series data. The processing unit 12 may execute processing of selecting a parameter from data of the first time-series date group and the second time-series date group combined into one and processing of individually selecting a parameter from the first time-series data group and the second time-series data group.

The processing unit 12 may generate a learned model of the entire common part (i.e., selected parameters from the first time-series data group and the second time-series data group) using the parameter selected for the entire time-series data. The processing unit 12 may generate a learned model of the individual medical institution by using the parameter selected for the individual time-series data. The processing unit 12 can improve the prediction accuracy of outcome in each medical institution by combining the learned model of the common part and the learned model of the individual medical institution. Combining the learned models includes, for example, taking a majority of a plurality of learned models and taking a weighted average.

Grouping of Time-Series Data

In an embodiment, the information processing device 10 may be configured to further receive an input of additional information including at least one of a disease, an initial symptom, or an individual attribute for each patient included in the plurality of patients by the input unit 11. The disease may include, for example, diseases such as cerebral infarction and heart failure. The initial symptom may include, for example, vital value data such as when the patient visits the emergency department and when the patient enters the ICU. The initial symptom may include, for example, information of a severity score. The severity score can include, for example, indices for severity assessment such as SAPS (2nd simplified acute physiology score) II and APACHE (Acute Physiology and Chronic Health Evaluation) II. The individual attributes may include, for example, gender and age, race, presence or absence of transport by an ambulance, and admission path.

The processing unit 12 may execute processing of grouping the time-series data into a plurality of groups on the basis of the additional information and selecting a parameter for each group among the plurality of groups. When the contents of the disease, the initial symptoms, the severity, and the like are different, the parameters to be acquired and the contents of the treatment are different. For example, for patients with heart failure, a model of treatment of heart failure is applied. In addition, the treatment strategy for patients with heart failure can vary depending on the blood pressure value of the initial symptom. Therefore, the processing unit 12 can collect time-series data of patients having a common disease, similar symptoms, and the like by grouping the time-series data by the additional information.

The processing unit 12 can execute processing such as calculation of an acquisition rate and an acquisition frequency of a parameter, selection of a parameter, and generation of training data on the grouped time-series data. By grouping time-series data of patients having a common disease, similar symptoms, and the like, a ratio is increased at which information such as administration information and a vital value of a medicine specific to the disease, symptoms, and the like can be collected. As a result, it can be expected that a combination of parameters highly correlated with an outcome (prognosis) can be selected, and missing of data of parameters used for training data can be reduced. In addition, by limiting the time-series data to be grouped to time-series data according to a specific disease or symptom, it can be expected that the prediction accuracy by the learned model generated using the training data becomes relatively high.

Period for Calculating Acquisition Frequency

In one embodiment, the processing unit 12 can increase the period for calculating the acquisition rate and the acquisition frequency of the parameter with the lapse of time. For example, the vital value of a patient entering the ICU may be frequently measured because the value is not stable immediately after entry. As the numerical value stabilizes with the lapse of time, the measurement interval of the vital value becomes relatively longer. Therefore, the period for calculating the acquisition rate and the acquisition frequency of the parameter can be lengthened with the lapse of time, for example, immediately after entering the ICU, one hour after entering the ICU, three hours after entering the ICU, one day after entering the ICU, and three days after entering the ICU.

Since there are many types of parameters to be acquired clinically and the acquisition frequency is relatively high for a while after entering the ICU, it can be expected that a relatively highly accurate learned model can be generated even in a relatively short period of time. On the other hand, when the period after entering the ICU increases, the type and number of parameters to be clinically acquired decrease, and thus, a combination of parameters different from those immediately after entering the ICU may be selected.

### First Example of Information Processing Method

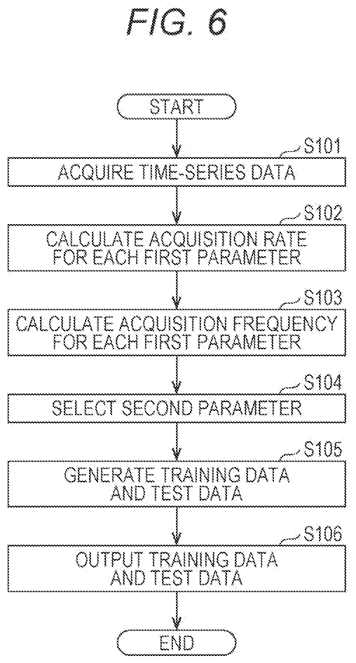

An example of an information processing method executed by the information processing device 10 according to an embodiment will be described with reference to FIG. 6. FIG. 6 illustrates a flow of information processing executed by the processing unit 12 of the information processing device 10. This processing can be executed by a processor included in the information processing device 10 according to a program. Such a program can be stored in a non-transitory computer-readable medium. Non-transitory computer-readable medium can include, for example, but is not limited to, magnetic storage medium, magneto-optical storage medium, semiconductor memory, and the like.

As shown in the flowchart of FIG. 6, the processing unit 12 of the information processing device 10 includes a time-series data acquiring unit 21, a parameter acquisition rate calculating unit 22, a parameter acquisition frequency calculating unit 23, a parameter selecting unit 24, and a training data generating unit 25. The processing unit 12 may not include the model generating unit 26 and the model evaluating unit 27.

First, the processing unit 12 acquires clinical time-series data regarding a plurality of patients via the input unit 11 (S101).

The processing unit 12 calculates an acquisition rate for each parameter (first parameter) included in the plurality of sets of time-series data acquired in S101 (S102).

The processing unit 12 calculates an acquisition frequency for each parameter (first parameter) included in the plurality of sets of time-series data acquired in S101 (S103).

The processing in S102 and S103 may be executed substantially simultaneously and in parallel. Further, S103 may be executed before S102.

The processing unit 12 selects a parameter (second parameter) to be adopted for the training data from among the parameters included in the time-series data on the basis of the acquisition rate and the acquisition frequency for each parameter (S104).

The processing unit 12 generates training data and test data for machine learning by using the data of the selected parameter among the plurality of sets of time-series data (S105).

The processing unit 12 outputs the generated training data and test data to another device or a storage medium via the output unit 13 in order to use the generated training data and test data in machine learning (S106).

The processing unit 12 may output only the information of the parameter selected in S104 to the outside in the next S106 without executing S105. In this case, another device generates training data and test data for machine learning.

### Second Example of Information Processing Method

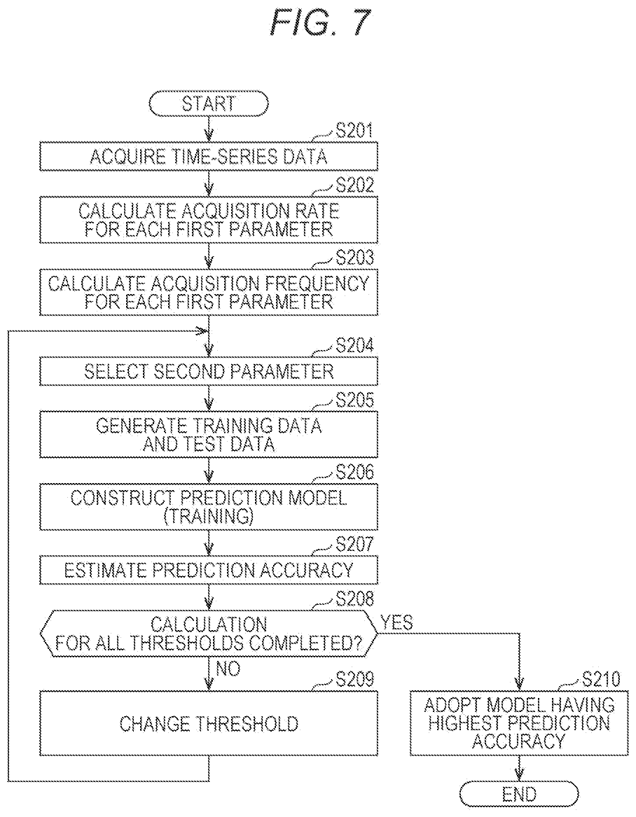

An example of an information processing method executed by the information processing device 10 according to another embodiment will be described with reference to FIG. 7. Since the processing from S201 to S205 in the flowchart of FIG. 7 is the same as or similar to the processing from S101 to S105 in FIG. 6, the description of the contents common to the description of S101 to S105 is omitted. As shown in the flowchart of FIG. 7, the processing unit 12 includes a model generating unit 26 and a model evaluating unit 27.

In S201 to S205, similarly to S101 to S105, the processing unit 12 executes processing of generating training data and test data for machine learning on the basis of the clinical time-series data acquired from the input unit 11. However, in S204, a plurality of thresholds is prepared, and a parameter is selected for one of the thresholds. The plurality of thresholds can be rephrased as provisional thresholds.

After generating the training data and the test data in S205, the processing unit 12 constructs a patient prognosis prediction model, which is a learned model by machine learning, using the generated training data (S206).

The processing unit 12 estimates the prediction accuracy by the prognosis prediction model constructed in S206 using the test data generated in S205 (S207). The processing unit 12 stores the prediction accuracy in the storage unit 14 in association with the provisional threshold.

In a case where the calculation from S204 to S207 for all the plurality of provisional thresholds has not been completed (S208: No), the processing unit 12 changes the threshold to a provisional threshold for which calculation has not been performed yet (S209).

After S209, the processing unit 12 returns to S204 and repeats the processing from S204 to S207.

In a case where the calculation from S204 to S207 is completed for all the plurality of provisional thresholds (S208: Yes), the processing unit 12 adopts the prognosis prediction model having the highest prediction accuracy stored in the storage unit 14 (S210), and ends the processing.

By doing so, the processing unit 12 can select the thresholds of the acquisition rate and the acquisition frequency at which high prediction accuracy can be obtained in order to select the parameter.

As described above, the information processing device 10 calculates the acquisition rate and the acquisition frequency of each parameter included in the plurality of sets of time-series data, and selects the parameter to be used for the training data using at least one of the calculated acquisition rate and acquisition frequency. As a result, it is possible to rather easily generate a required number of sets of training data with relatively high quality for performing machine learning from the patient clinical time-series data. In addition, this makes it relatively easy to generate training data for machine learning from the clinical data.

Further, in the above embodiment, a parameter in which at least one of the acquisition rate and the average acquisition frequency exceeds the threshold can be selected, and the training data can be generated using the time-series data having the data in which the selected parameter exceeds the threshold. As a result, it is possible to generate training data with less data missing, and it is possible to expect improvement in accuracy of machine learning. In addition, the processing load can be reduced because the processing of estimating the missing part of the data is not required or can be reduced in order to correct the missing of the training data.

Although the above-described embodiments have been described as representative examples, it will be apparent to those skilled in the art that many modifications and substitutions are possible within the spirit and scope of the present disclosure. Therefore, the present disclosure should not be construed as limited by the above-described embodiments, and various modifications and changes can be made without departing from the scope of the claims. For example, functions and the like included in each component, each step or process, or the like can be rearranged so as not to be logically inconsistent, and a plurality of components, steps, processes and the like can be combined into one or divided.

What is claimed is:

1. An information processing device used in a system that predicts prognosis of a patient by machine learning, the information processing device comprising:
an input unit configured to receive an input of a plurality of sets of time-series data corresponding to a plurality of patients, the plurality of sets of time-series data including a plurality of first parameters related to at least one of a condition and a treatment of each of the plurality of patients; and
a processing unit configured to calculate an acquisition rate and an acquisition frequency of each of the plurality of first parameters included in the plurality of sets of time-series data, and to select a second parameter to be used for training data from the plurality of first parameters by using at least one of the calculated acquisition rate and the calculated acquisition frequency.

2. The information processing device according to claim 1, wherein the acquisition rate indicates a ratio at which the first parameter is included in the plurality of sets of time-series data.

3. The information processing device according to claim 1, wherein the acquisition frequency indicates a frequency at which the first parameter is included in the plurality of sets of time-series data within a predetermined period of the plurality of sets of time-series data.

4. The information processing device according to claim 1, wherein in a case where the at least one of the acquisition rate and the acquisition frequency of the first parameter exceeds a predetermined threshold, the processing unit is configured to select the first parameter as the second parameter to be used for the training data.

5. The information processing device according to claim 4, wherein a threshold of the acquisition frequency is different among the plurality of first parameters.

6. The information processing device according to claim 4, wherein the threshold of the acquisition frequency is determined on a basis of a number of sets of the plurality of sets of time-series data that include the first parameter and exceed the threshold.

7. The information processing device according to claim 1, wherein the plurality of sets of time-series data includes a first time-series data group and a second time-series data group, and the processing unit is configured to execute processing of selecting the second parameter from data of the first time-series date group and the second time-series data group combined into one, and processing of individually selecting the second parameter from the first time-series data group and the second time-series data group.

8. The information processing device according to claim 1, wherein
the input unit is configured to receive an input of additional information including at least one of an initial symptom, an individual attribute, or a disease for each patient among the plurality of patients; and
the processing unit is configured to group the time-series data into a plurality of groups on a basis of the input of additional information and to select the second parameter for each group among the plurality of groups.

9. The information processing device according to claim 3, wherein the processing unit is configured to increase the predetermined period for calculating the acquisition frequency as time elapses.

10. The information processing device according to claim 1, wherein the processing unit is configured to generate training data by using the selected second parameter.

11. The information processing device according to claim 10, wherein the processing unit is configured to generate the training data in a data format based on the acquisition frequency of the selected second parameter.

12. The information processing device according to claim 10, wherein the processing unit is configured to generate a learned model for predicting prognosis of a patient using the training data.

13. The information processing device according to claim 1, wherein for each of a plurality of provisional thresholds, in a case where the at least one of the acquisition rate and the acquisition frequency of the first parameter exceeds the provisional threshold, the processing unit is configured to select the first parameter as a provisional parameter to be used for the training data, to generate the training data and test data using the provisional parameter, to generate a learned model for predicting prognosis of a patient using the training data, performs processing of determining accuracy of the learned model using the test data, and to select the provisional parameter with the determined highest accuracy as the second parameter.

14. The information processing device according to claim 1, wherein the plurality of sets of time-series data includes at least any one of administration information of a medicine, a vital value, examination information, finding information, water intake information, water loss information, and treatment information.

15. The information processing device according to claim 14, wherein
the administration information of the medicine includes at least one of information of a type, an administration route, a dose, and an administration rate of an administration medicine;
the vital value includes at least one of information of a body temperature, a blood pressure, a heart rate, a respiratory rate, a pulse rate, oxygen saturation, a weight value, a central venous pressure, and an oxygen concentration during inhalation;
the examination information includes at least one of information of blood examination data, blood gas data, a urine examination, an electrocardiogram, and a diagnostic imaging result;
the finding information includes at least one of information of congestion, cyanosis, and a level of consciousness;
the water intake information includes at least one of information of a water intake amount and an infusion amount;
the water loss information includes at least one of information of a urine amount and a blood loss amount; and
the treatment information includes at least one of information of introduction of a dialysis device, disengagement of the dialysis device, setting of the dialysis device, introduction of a ventilator, disengagement of the ventilator, and setting of the ventilator.

16. An information processing method executed by an information processing device used in a system that predicts prognosis of a patient by machine learning, the information processing method comprising:
acquiring a plurality of sets of time-series data corresponding to a plurality of patients, the plurality of sets of time-series data including a plurality of first parameters related to at least one of a condition and a treatment of each of the patients;
calculating an acquisition rate and an acquisition frequency of each of the first parameters included in the plurality of sets of time-series data; and
selecting a second parameter to be used for training data from the plurality of first parameters using at least one of the calculated acquisition rate and the calculated acquisition frequency.

17. The information processing method according to claim 16, wherein the acquisition rate indicates a ratio at which the first parameter is included in the plurality of sets of time-series data.

18. The information processing method according to claim 16, wherein the acquisition frequency indicates a frequency at which the first parameter is included in data within a predetermined period of the plurality of sets of time-series data.

19. The information processing method according to claim 16, wherein in a case where the at least one of the acquisition rate and the acquisition frequency of the first parameter exceeds a predetermined threshold, the method includes:
selecting the first parameter as the second parameter to be used for the training data.

20. A non-transitory computer-readable medium storing a computer program for causing an information processing device to execute information processing executed by the information processing device used in a system that predicts prognosis of a patient by machine learning, the information processing comprising:
acquiring a plurality of sets of time-series data corresponding to a plurality of patients, the plurality of sets of time-series data including a plurality of first parameters related to at least one of a condition and a treatment of each of the patients;
calculating an acquisition rate and an acquisition frequency of each of the first parameters included in the plurality of sets of time-series data; and
selecting a second parameter to be used for training data from the plurality of first parameters using at least one of the calculated acquisition rate and the calculated acquisition frequency.
